(19) United States
(12) Patent Application Publication
(10) Pub. No.: US 2004/0053235 A1
(43) Pub. Date: Mar. 18, 2004

(54) Gene sequence

(76) Inventors: Nicholas Smirnoff, Exeter (GB); Glen Wheeler, Exeter (GB)

Correspondence Address: SHERIDAN ROSS PC, 1560 BROADWAY, SUITE 1200, DENVER, CO, 80202

(21) Appl. No.: 10/240,136

(22) PCT Filed: Mar. 29, 2001
(86) PCT No.: PCT/GB01/01412

(30) Foreign Application Priority Data
Mar. 29, 2000 (GB) 0007651.3

(51) Int. Cl. C12Q 1/68; C07H 21/04; C12N 9/02; C12P 21/02; C12N 5/06

(52) U.S. Cl. 435/6; 435/69.1; 435/189; 435/320.1; 435/325; 536/23.2

(57) ABSTRACT

Disclosed are isolated L-galactose dehydrogenase proteins and biologically active homologues thereof, as well as nucleic acid molecules encoding such proteins. Also disclosed are methods of producing L-galactose dehydrogenase, and genetically modified organisms having increased L-galactose dehydrogenase action. Methods of producing L-ascorbic acid or esters thereof using such genetically modified organisms are disclosed.

### FIELD OF INVENTION

This invention relates to L-galactose dehydrogenase proteins (L-gal DH) and nucleic acid sequences encoding L-galactose dehydrogenase proteins, and to methods of making and using L-galactose dehydrogenase nucleic acid molecules and proteins.

### BACKGROUND

Nearly all forms of life, both plant and animal, either synthesize L-ascorbic acid (also referred to herein as “ascorbic acid”), commonly known as Vitamin C, or require it as a nutrient Ascorbic acid was first identified to be useful as a dietary supplement for humans and animals for the prevention of scurvy. Ascorbic acid, however, affects human physiological functions such as the adsorption of iron, cold tolerance, the maintenance of the adrenal cortex, wound healing, the synthesis of polysaccharides and collagen, the formation of cartilage, dentine, bone and teeth, the maintenance of capillaries, and is useful as an antioxidant.

L-ascorbic acid is a major metabolite of plants reaching concentrations of up to 5 mM, in leaf tissues. It functions as an antioxidant and also has proposed roles in photosynthesis, trans-membrane electron transport, cell expansion and cell division (Smirnoff, (1996) Ann Bot 78, 661-669, Noctor and Foyer, (1998) Ann. Rev. Plant Physiol. Plant Mol. Biol. 49, 249-279; Smirnoff and Wheeler, (1999) Ascorbic acid metabolism in plants. In “Plant Carbohydrate Biochemistry” [J. A. Bryant, M. M. Burrell and N. J. Kruger, Eds.], pp. 215-229. Bios Scientific Publishers, Oxford).

Humans are unable to synthesize vitamin C themselves, and as a consequence, plant derived ascorbate acts a major source of this essential antioxidant in their diet. A lack of ascorbic acid in the human diet can cause scurvy, a condition caused by impaired collagen biosynthesis (Davies et al., (1991) “Vitamin C: its Chemistry and Biochemistry. ” Royal Society of Chemistry, Cambridge).

Plants are able to synthesize ascorbic acid from D-glucose-6-phosphate through the following intermediates: D-glucose-6-phosphate; D-fructose-6-phosphate; D-mannose-6-phosphate; D-mannose-1-phosphate; GDP-D-mannose; GDP-L-galactose; L-galactose-1-phosphate; L-galactose; L-galactono-1,4-lactone and L-ascorbic acid (Wheeler et al., (1998) Nature 393, 365-369). Manipulation of one or all of the genes that encode for the enzymes facilitating these steps could lead to the production of transgenic organisms capable of overproducing ascorbic acid. Transgenic plants would have increased nutritional value and may also be more stress tolerant (Smirnoff, (1996) Ann Bot 78, 661-669, Noctor and Foyer, (1998) Ann. Rev. Plant Physiol. Plant Mol. Biol. 49, 249-279) whereas transgenic micro-organisms could be used to produce ascorbic acid biologically rather than by current processes of chemical synthesis. The enzyme catalyzing the penultimate step of the ascorbate biosynthetic pathway in plants has been identified as L-galactose dehydrogenase. This enzyme is responsible for oxidizing L-galactose to L-galactono-1,4-lactone (Wheeler et al., (1998) Nature 393, 365-369). L-galactose dehydrogenase has been purified to homogeneity and an N-terminal amino acid sequence has been obtained from the purified enzyme in Pisum sativum (PCT Publication No. WO 99/33995, published Jul. 8, 1999, incorporated herein by reference in its entirety). However, prior to the present invention, the complete amino acid sequence of the L-galactose dehydrogenase enzyme and the nucleic acid sequence encoding such an enzyme, had not been identified.

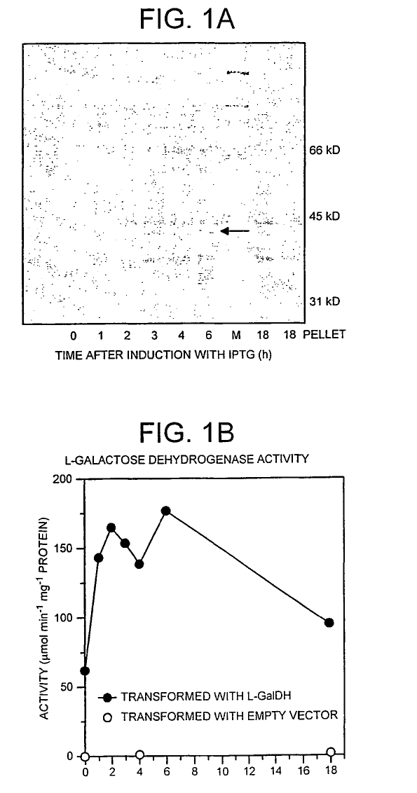

FIG. 1A is a line graph showing L-galactose dehydrogenase activity in E. coli transformed with a recombinant nucleic acid molecule encoding Arabidopsis thaliana L-galactose dehydrogenase.

### SUMMARY OF INVENTION

This invention relates to an enzyme, L-galactose dehydrogenase, to nucleic acid sequences encoding such a protein, to amino acid sequences of such a protein, and to methods of making and using a protein having L-galactose dehydrogenase biological activity. L-galactose dehydrogenase catalyzes a reaction in the biosynthetic pathway of L-ascorbic acid (L-threo-2-hexenono-1,4-lactone) in plants (Wheeler et al., (1998), Nature 393, 365-369). The present inventors have determined a nucleotide sequence encoding L-galactose dehydrogenase in plants and have demonstrated that a nucleic acid molecule comprising this nucleic acid sequence, when expressed in E. coli, produces an enzyme that has L-galactose dehydrogenase biological activity. This nucleic acid sequence can therefore be utilized to make transgenic organisms with an enhanced ability to synthesize ascorbic acid. One embodiment of the present invention relates to an isolated protein having L-galactose dehydrogenase biological activity. According to the present invention, isolated proteins having L-galactose dehydrogenase biological activity can be identified in a straight-forward manner by the ability of the protein to catalyze the conversion of L-galactose to L-galactono-1,4-lactone. In addition, a protein having L-galactose dehydrogenase biological activity is typically NAD or NADP-dependent, and oxidizes carbon atom 1 of L-galactose. L-galactose dehydrogenase biological activity can be detected using an enzyme assay that measures such biological activity. Such an assay for L-galactose dehydrogenase is described, for example, in Example 2 in PCT Publication No. WO 99/33995, published Jul. 8, 1999, incorporated herein by reference in its entirety.

According to the present invention, an isolated protein having L-galactose dehydrogenase biological activity includes a naturally occurring L-galactose dehydrogenase protein, including full-length proteins and truncated proteins having L-galactose dehydrogenase biological activity, as well as fusion proteins and homologues of naturally occurring L-galactose dehydrogenase proteins. The term “isolated L-galactose dehydrogenase” or “isolated protein having L-galactose dehydrogenase biological activity”, refers to a protein having L-galactose dehydrogenase activity which is outside of its natural environment in a pure enough form to achieve a significant increase in activity over crude extracts having L-galactose dehydrogenase activity. Such an isolated protein having L-galactose dehydrogenase biological activity can include, but is not limited to, purified L-galactose dehydrogenase (See, for example, PCT Publication No. WO 99/33995, ibid.) and a recombinantly produced L-galactose dehydrogenase, including recombinantly produced naturally occurring L-galactose dehydrogenase proteins (See Example 1) and recombinantly or synthetically produced homologues thereof.

According to the present invention, a homologue of an L-galactose dehydrogenase protein includes L-galactose dehydrogenase proteins which differ from naturally occurring L-galactose dehydrogenase proteins by at least one or a few, but not limited to one or a few, amino acids deletions (e.g., a truncated version of the protein, such as a peptide), insertions, inversions, substitutions and/or derivatizations (e.g., by glycosylation, phosphorylation, acetylation, myristoylation, prenylation, palmitation, amidation and/or addition of glycosylphosphatidyl inositol). According to the present invention, a homologue of a naturally occurring L-galactose dehydrogenase protein has measurable (i.e., detectable using standard techniques) L-galactose dehydrogenase biological activity, such activity being described above. In another embodiment, a homologue of a naturally occurring L-galactose dehydrogenase protein can also be identified as a protein having at least one epitope which elicits an immune response against a protein having an amino acid sequence selected from the group of SEQ ID NO:1 and SEQ ID NO:5. In another embodiment, a homologue of an L-galactose dehydrogenase protein is a protein having an amino acid sequence that is sufficiently similar to a naturally occurring L-galactose dehydrogenase amino acid sequence that a nucleic acid sequence encoding the homologue is capable of hybridizing under low, moderate, or high stringency conditions to (i.e., with) a nucleic acid molecule encoding the natural L-galactose dehydrogenase (i.e., to the complement of the nucleic acid strand encoding the natural L-galactose dehydrogenase amino acid sequence). This aspect of the present invention will be described in detail below. A nucleic acid sequence complement of nucleic acid sequence encoding an L-galactose dehydrogenase of the present invention refers to the nucleic acid sequence of the nucleic acid strand that is complementary to (i.e., can form a complete double helix with) the strand which encodes the L-galactose dehydrogenase.

It will be appreciated that a double stranded DNA which encodes a given amino acid sequence comprises a single strand DNA and its complementary strand having a sequence that is a complement to the single strand DNA. As such, nucleic acid molecules which encode the L-galactose dehydrogenase of the present invention can be either double-stranded or single-stranded, and include those nucleic acid molecules that form stable hybrids under low, moderate or high stringency conditions with a nucleic acid sequence that encodes the amino acid sequence selected from the group consisting of SEQ ID NO:1 or SEQ ID NO:5, and/or with the complement of the nucleic acid that encodes amino acid sequence selected from the group of SEQ ID NO:1 or SEQ ID NO:5. Methods to deduce a complementary sequence are known to those skilled in the art. It should be noted that since amino acid sequencing and nucleic acid sequencing technologies are not entirely error-free, the sequences presented herein, at best, represent apparent sequences of L-galactose dehydrogenases of the present invention.

L-galactose dehydrogenase homologues can be the result of natural allelic variation or natural mutation. L-galactose dehydrogenase homologues of the present invention can also be produced using techniques known in the art including, but not limited to, direct modifications to the naturally occurring protein or modifications to the nucleic acid sequence encoding the naturally occurring protein using, for example, classic or recombinant DNA techniques to effect random or targeted mutagenesis. A naturally occurring allelic variant of a nucleic acid encoding L-galactose dehydrogenase is a gene that occurs at essentially the same locus (or loci) in the genome as the gene which encodes a naturally occurring L-galactose dehydrogenase, such as the genes encoding a protein comprising an amino acid sequence selected from the group of SEQ ID NO:1 or SEQ ID NO:5, but which, due to natural variations caused by, for example, mutation or recombination, has a similar but not identical sequence. Natural allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

L-galactose dehydrogenase proteins also include expression products of gene fusions (for example, used to overexpress soluble, active forms of the recombinant enzyme), of mutagenized genes (such as genes having codon modifications to enhance gene transcription and translation), and of truncated genes (such as genes having signal sequences removed which are poorly tolerated in a particular recombinant host). Although proteins useful in the present invention preferably have L-galactose dehydrogenase biological activity, it is noted that L-galactose dehydrogenase proteins and protein homologues which do not have L-galactose dehydrogenase enzymatic activity are also envisioned by the present inventors. Such proteins are useful, for example, for the production of antibodies against L-galactose dehydrogenase proteins for use in purification and/or identification of L-galactose dehydrogenase proteins, which are in turn useful in the methods of the present invention.

The minimal size of a protein and/or homologue of the present invention is a size sufficient to have L-galactose dehydrogenase biological activity. Preferably, such a protein includes at least an NAD or an NADP binding site, a site sufficient to catalyze the conversion of L-galactose to L-galactono-1,4-lactone, and particularly, to oxidize carbon atom 1 of L-galactose. Determination of, such minimum portions is well within the ability of one skilled in the art. For example, now that the nucleic acid and amino acid sequence of L-galactose dehydrogenase is known, one can compare the sequence to other dehydrogenase enzymes for which the active site is known to provide information useful in estimating the active site of the present L-galactose dehydrogenase enzyme. Additionally, one of skill in the art can easily perform mutational analyses, including analysis of truncated forms of the protein, using the knowledge of the sequence provided by the present invention and the L-galactose dehydrogenase enzyme assay described herein.

In one embodiment, the minimal size of a protein and/or homologue of the present invention is a size sufficient to be encoded by a nucleic acid molecule capable of forming a stable hybrid with the complementary sequence of a nucleic acid molecule encoding the corresponding natural protein. As such, the size of the nucleic acid molecule encoding such a protein is dependent on nucleic acid composition and percent homology between the nucleic acid molecule and complementary sequence as well as upon hybridization conditions per se (e.g., temperature, salt concentration, and formamide concentration). The minimal size of such nucleic acid molecules is typically at least about 24 to about 960 nucleotides in length. There is no limit, other than a practical limit, on the maximal size of such a nucleic acid molecule in that the nucleic acid molecule can include a portion of a coding region sufficient to encode a protein having L-galactose dehydrogenase biological activity, an entire coding region, or an entire gene. Similarly, the minimal size of L-galactose dehydrogenase protein or homologue of the present invention is from about 8 to 319 amino acids in length, with preferred sizes depending on whether a full-length, multivalent (i.e., fusion protein having more than one domain, each of which has a function), or functional portions of such proteins are desired.

A preferred protein having L-galactose dehydrogenase biological activity of the present invention include proteins which comprise an amino acid sequence having at least about 40%, and preferably, at least about 50%, and more preferably, at least about 60%, and more preferably, at least about 70%, and more preferably, at least about 80%, and even more preferably, at least about 90% identity with an amino acid sequence selected from SEQ ID NO:1 and/or SEQ ID NO:5. As used herein, reference to a percent (%) identity refers to a BLAST homology search with the default parameters such as those identified in Table 1. All references to percent identity discussed herein were determined using BLAST Version 2.0. BLAST parameters and all references cited within such parameters are publicly available at http://www.ncbi.nlm.nih.gov/blast.

### Table 1

### BLAST Search Parameters

Histogram

Display a histogram of scores for each search; default is yes. (See parameter H in the BLAST Manual).

Descriptions

Restricts the number of short descriptions of matching sequences reported to the number specified; default limit is 100 descriptions. (See parameter V in the manual page). See also EXPECT and CUTOFF.

Alignments

Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

Expect

The statistical significance threshold for reporting matches against database sequences; the default value is 10, such that 10 matches are expected to be found merely by chance, according to the stochastic model of Karlin and Altschul (1990).

If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

Cutoff

Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

Matrix

Specify an alternate scoring matrix for BLASTP, BLASTX, TBLASTN and TBLASTX The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

Strand

Restrict a TBLASTN search to just the top or bottom strand of the database sequences; or restrict BLASTN, BLASTX or TBLASIX search to just reading frames on the top or bottom strand of the query sequence.

Filter

Mask off segments of the query sequence that have low compositional complexity, as determined by the SEG program of Wootton & Federhen (Computers and Chemistry, 1993), or segments consisting of short-periodicity internal repeats, as determined by the SNU program of Clayerie & States (Computers and Chemistry, 1993), or, for BLASTN, by the DUST program of Tatusov and Lipman (in preparation). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN” and the letter “X” in protein sequences (e.g., “XXXXXXXXX” Users may turn off filtering by using the “Filter” option on the “Advanced options for the BLAST server” page.

Filtering is only applied to the query sequence (or its translation products), not to database sequences. Default filtering is DUST for BLASTN, SEG for other programs.

It is not unusual for nothing at all to be masked by SEG, SNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect

NCBI-gi

Causes NCBI gi identifiers to be shown in the output, in addition to the accession and/or locus name.

According to one embodiment of the present invention, homology or percent identity between two or more nucleic acid or amino acid sequences is performed using other methods known in the art for aligning and/or calculating percentage identity. To compare the homology/percent identity between two or more sequences as set forth above, for example, a module contained within DNASTAR (DNASTAR, Inc., Madison, Wis.) can be used. In particular, to calculate the percent identity between two nucleic acid or amino acid sequences, the Lipman-Pearson method, provided by the MegAlign module within the DNASTAR program, can be used, with the following parameters, also referred to herein as the Lipman-Pearson standard default parameters:

(1) Ktuple=2;

(2) Gap penalty=4;

(3) Gap length penalty=12.

According to another embodiment of the present invention, to align two or more nucleic acid or amino acid sequences, for example to generate a consensus sequence or evaluate the similarity at various positions between such sequences, a CLUSTAL alignment program (e.g., CLUSTAL, CLUSTAL V, CLUSTAL W), also available as a module within the DNASTAR program, can be used using the following parameters, also referred to herein as the CLUSTAL standard default parameters:

Multiple Alignment Parameters (i.e., for More Than 2 Sequences):

(1) Gap penalty=10;

(2) Gap length penalty=10;

Pairwise Alignment Parameters (i.e., for Two Sequences):

(1) Ktuple=1;

(2) Gap penalty=3;

(3) Window=5;

(4) Diagonals saved=5.

In one embodiment, an isolated protein having L-galactose dehydrogenase biological activity of the present invention has an amino acid sequence which includes at least 8 consecutive amino acids, and more preferably, at least 12 consecutive amino acids, and even more preferably, at least 16 consecutive amino acids of SEQ ID NO:1 or SEQ ID NO:5. More preferably, an isolated protein having L-galactose dehydrogenase biological activity of the present invention has an amino acid sequence which includes at least 32 consecutive amino acids, and more preferably, at least about 64 consecutive amino acids, and even more preferably, at least 128 consecutive amino acids, and even more preferably, at least 256 consecutive amino acids of SEQ ID NO:5. According to the present invention, the term “consecutive” means to be connected in an unbroken sequence. For a first sequence to have “100% identity with” or “X consecutive amino acids (or nucleotides) of” a second sequence means that the first sequence exactly matches the second sequence over the recited number of amino acids (or nucleotides) with no gaps between amino acids (or nucleotides).

In one embodiment, a protein having L-galactose dehydrogenase biological activity is encoded by a nucleic acid molecule comprising a nucleic acid sequence that hybridizes under low stringency conditions to a nucleic acid sequence represented by SEQ ID NO:4. Preferably, a protein having L-galactose dehydrogenase biological activity is encoded by a nucleic acid molecule comprising a nucleic acid sequence that hybridizes under moderate stringency conditions, and more preferably, under high stringency conditions, to a nucleic acid sequence represented by SEQ ID NO:4. In one embodiment of the present invention a protein having L-galactose dehydrogenase biological activity is encoded by a nucleic acid molecule comprising a nucleic acid sequence represented by SEQ ID NO:4. SEQ ID NO:4 is a nucleic acid sequence which represents the full-length coding region of an Arabidopis thaliana L-galactose dehydrogenase of the present invention.

As used herein, stringent hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., (1984), Anal. Biochem. 138, 267-284; Meinkoth et al., ibid., is incorporated by reference herein in its entirety.

More particularly, low stringency hybridization and washing conditions, as referred to herein, refer to conditions which permit isolation of nucleic acid molecules having at least about 40% nucleic acid sequence identity with the nucleic acid molecule being used to probe in the hybridization reaction (i.e., conditions permitting about 60% or less mismatch of nucleotides). Moderate stringency hybridization and washing conditions, as referred to herein, refer to conditions which permit isolation of nucleic acid molecules having at least about 55% nucleic acid sequence identity with the nucleic acid molecule being used to probe in the hybridization reaction (i.e., conditions permitting about 45% or less mismatch of nucleotides). High stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). As discussed above, one of skill in the art can use a reference such as Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, hybridization conditions for DNA:DNA hybrids include hybridization at an ionic strength of 6×SSC (0.9 M Na+) at a temperature of between about 20° C. and about 35° C., more preferably, between about 28° C. and about 40° C., and even more preferably, between about 35° C. and about 45° C. In particular embodiments, hybridization conditions for DNA:RNA hybrids include hybridization at an ionic strength of 6×SSC (0.9 M Na+) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62.

Particularly preferred L-galactose dehydrogenase proteins of the present invention include proteins which comprise an amino acid sequence selected from SEQ ID NO: 1 and SEQ ID NO:5. SEQ ID NO:1 represents a 19 amino acid N-terminal sequence of an L-galactose dehydrogenase from Pisum sativum (See WO 99/33995, ibid.). SEQ ID NO:5 represents the amino acid sequence of a full-length L-galactose dehydrogenase from Arabidopsis thaliana (See Example 1).

The present invention also includes a fusion protein that includes an L-galactose dehydrogenase-containing domain attached to one or more fusion segments. Suitable fusion segments for use with the present invention include, but are not limited to, segments that can: enhance a protein's stability; provide other enzymatic activity; and/or assist purification of a L-galactose dehydrogenase (e.g., by affinity chromatography). A suitable fusion segment can be a domain of any size that has the desired function (e.g., imparts increased stability, solubility, action or activity; provides other enzymatic activity; and/or simplifies purification of a protein). Fusion segments can be joined to amino and/or carboxyl termini of the L-galactose dehydrogenase-containing domain of the protein and can be susceptible to cleavage in order to enable straight-forward recovery of an L-galactose dehydrogenase. Fusion proteins are preferably produced by culturing a recombinant cell transformed with a fusion nucleic acid molecule that encodes a protein including the fusion segment attached to either the carboxyl and/or amino terminal end of an L-galactose dehydrogenase-containing domain.

L-galactose dehydrogenase proteins can be isolated from a various plants, including higher plants, such as Pisum sativum and Arabidopsis thaliana, as well as microalgae, including species of Prototheca or Chlorella. A particularly preferred L-galactose dehydrogenase is a Pisum sativum or a Arabidopsis thaliana L-galactose dehydrogenase.

One embodiment of the present invention relates to a recombinant nucleic acid molecule comprising an expression vector operatively linked to a nucleic acid molecule comprising a nucleic acid sequence encoding a protein having L-galactose dehydrogenase biological activity, such protein being described in detail above. As discussed above, a protein having L-galactose dehydrogenase biological activity can include naturally occurring L-galactose dehydrogenase proteins and homologues thereof, which include proteins which differ from naturally occurring L-galactose dehydrogenase proteins by at least one or a few, but not limited to one or a few, amino acids deletions (e.g., a truncated version of the protein, such as a peptide), insertions, inversions, substitutions and/or derivatizations. In a preferred embodiment, a nucleic acid molecule useful in a recombinant nucleic acid molecule of the present invention encodes a protein that is at least about 40% identical, and more preferably, at least about 50%, and more preferably, at least about 60%, and more preferably, at least about 70%, and more preferably, at least about 80%, and even more preferably, at least about 90% identical to an amino acid sequence selected from SEQ ID NO:1 and/or SEQ ID NO:5. In one embodiment, a recombinant nucleic acid molecule of the present invention includes a nucleic acid molecule comprising a nucleic acid sequence that is at least about 97% identical to SEQ ID NO:4 over at least 27 nucleotides of SEQ ID NO:4. In one embodiment, of the present invention, a nucleic acid homologue (i.e., encoding a homologue of an L-galactose dehydrogenase protein) comprises a nucleic acid sequence that is less than 100% identical to a nucleic acid sequence represented by SEQ ID NO:4. Preferably, a nucleic acid molecule useful in a recombinant nucleic acid molecule of the present invention encodes a protein comprising an amino acid sequence selected from SEQ ID NO:1 or SEQ ID NO:5.

In one embodiment, a recombinant nucleic acid molecule of the present invention includes a nucleic acid molecule comprising a nucleic acid sequence comprising at least about 24 consecutive nucleotides of SEQ ID NO:4, and more preferably, at least about 48 consecutive nucleotides, and more preferably, at least about 96 consecutive nucleotides, and more preferably, at least about 192 consecutive nucleotides, and more preferably, at least about 384 consecutive nucleotides, and even more preferably, 768 consecutive nucleotides of SEQ ID NO:4. In another embodiment, a recombinant nucleic acid molecule of the present invention includes a nucleic acid molecule comprising a nucleic acid sequence encoding a protein comprising an amino acid sequence which includes at least 8 consecutive amino acids, and more preferably, at least 12 consecutive amino acids, and even more preferably, at least 16 consecutive amino acids of SEQ ID NO:1 or SEQ ID NO:5. More preferably, a recombinant nucleic acid molecule of the present invention includes a nucleic acid molecule comprising a nucleic acid sequence encoding a protein comprising an amino acid sequence which includes at least 32 consecutive amino acids, and more preferably, at least about 64 consecutive amino acids, and even more preferably, at least 128 consecutive amino acids, and even more preferably, at least 256 consecutive amino acids of SEQ ID NO:5.

In yet another embodiment, a recombinant nucleic acid molecule of the present invention includes a nucleic acid molecule comprising a nucleic acid sequence that hybridizes under low stringency conditions, and more preferably under moderate stringency conditions, and even more preferably under high stringency conditions with a nucleic acid sequence represented by SEQ ID NO:4. Such conditions have been described in detail above. In a preferred embodiment, a recombinant nucleic acid molecule of the present invention includes a nucleic acid molecule comprising a nucleic acid sequence represented by SEQ ID NO:4.

According to the present invention, a recombinant nucleic acid molecule includes at least one nucleic acid molecule encoding a protein having L-galactose dehydrogenase biological activity of the present invention, which is operatively linked to any expression vector capable of expressing the nucleic acid molecule in a suitable host cell. An expression vector is a DNA or RNA vector that is capable of transforming or transfecting a host cell and of effecting expression of a specified nucleic acid molecule, which in the present invention, is a nucleic acid molecule encoding a protein having L-galactose dehydrogenase biological activity. Preferably, the expression vector is also capable of replicating within the host cell. In the present invention, expression vectors are typically plasmids, although any expression vector suitable for expressing a nucleic acid molecule of the present invention in a host cell, and particularly, in a host cell of a live organism, is encompassed herein. Such a vector can contain nucleic acid sequences that are not naturally found adjacent to the isolated nucleic acid molecules to be inserted into the vector. Expression vectors can also be used in the cloning, sequencing, and/or otherwise manipulating of nucleic acid molecules. Particularly preferred expression vectors of the present invention include any vectors that are suitable for use in (i.e., function, direct gene expression) in a plant host cell, including, but not limited to, a higher plant host cell or a microalgal host cell (i.e., a plant expression vector), or a microorganism host cell, including, but not limited to, a bacterial host cell, a fungal host (e.g., a yeast) cell or a microalgal host cell (i.e., a microorganism expression vector).

The phrase, “operatively linked”, refers to insertion of a nucleic acid molecule into an expression vector in a manner such that the molecule is able to be expressed when transformed into a host cell. Nucleic acid molecules of the present invention can be operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in the host cell, and preferably include any transcription control sequence that can function in a plant host cell or a microorganism host cell. A variety of such transcription control sequences are known to those skilled in the art, and a few are exemplified in the Examples section below.

In accordance with the present invention, an isolated nucleic acid molecule, or a nucleic acid molecule suitable for use in a recombinant nucleic acid molecule of the present invention, is a nucleic acid molecule that has been removed from its natural milieu (i.e., that has been subject to human manipulation). As such, “isolated” does not reflect the extent to which the nucleic acid molecule has been purified. Preferably, the nucleic acid molecule, which encodes a protein having L-galactose dehydrogenase biological activity, does not include coding regions for other proteins (i.e., proteins other than L-galactose dehydrogenase) which flank or are located near (i.e., within 1-10,000 bp of) an L-galactose dehydrogenase gene in its natural milieu. An isolated nucleic acid molecule can include DNA, RNA, or derivatives of either DNA or RNA.

An isolated nucleic acid molecule of the present invention can be obtained from its natural source either as an entire (i.e., complete) gene or a portion thereof capable of forming a stable hybrid with that gene. An isolated nucleic acid molecule can also be produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect within the host cell. Preferably, a homologue of a nucleic acid sequence encodes a homologue of a protein having L-galactose dehydrogenase activity as described in detail above (i.e., a nucleic acid molecule homologue encodes a protein homologue of the present invention).

A nucleic acid molecule homologue can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein having L-galactose dehydrogenase biological activity.

Knowing the nucleic acid sequences of certain nucleic acid molecules of the present invention allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions). Such nucleic acid molecules can be obtained in a variety of ways, including traditional cloning techniques using oligonucleotide probes to screen appropriate libraries or DNA and PCR amplification of appropriate libraries or DNA using oligonucleotide primers. Techniques to clone and amplify genes are disclosed, for example, in Sambrook et al., ibid. Example 1 describes the cloning of a nucleic acid molecule encoding an L-galactose dehydrogenase protein of the present invention.

It may be appreciated by one skilled in the art that use of recombinant DNA technologies can improve expression of transformed or transfected nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively ‘inking nucleic’ acid molecules to high-copy number plasmids, integration of the nucleic acid molecules into the host cell chromosome, addition of vector stability sequences to plasmids, substitutions or modifications of transcription control signals (e.g., promoters, operators, enhancers), substitutions or modifications of translational control signals, modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

Transformation or transfection of a nucleic acid molecule into a cell can be accomplished by any method by which a nucleic acid molecule can be inserted into the cell. As used herein, the term “transformation” is typically used to refer to a permanent insertion of a recombinant nucleic acid molecule of the present invention into a genome of a host cell or organism. The term “transfection” is used to refer to a more transient insertion of a recombinant nucleic acid molecule of the present invention into a host cell or organism. Transformation and transfection techniques are well known to those of skill in the art.

According to the present invention, reference to an L-galactose dehydrogenase gene includes all nucleic acid sequences related to a natural L-galactose dehydrogenase gene such as regulatory regions that control production of the L-galactose dehydrogenase protein encoded by that gene (such as, but not limited to, transcription, translation or post-translation control regions) as well as the coding region itself. In another embodiment, an L-galactose dehydrogenase gene can be an allelic variant that includes a similar but not identical sequence to the nucleic acid sequence encoding a given L-galactose dehydrogenase. Allelic variants have been previously described above.

One embodiment of the present invention relates to a method to produce a protein having L-galactose dehydrogenase biological activity. The method includes the step of culturing a cell that has been genetically modified to express a recombinant nucleic acid molecule encoding a protein having L-galactose dehydrogenase biological activity as described in detail above, under conditions whereby the protein encoded by the recombinant nucleic acid molecule is expressed (i.e., produced, translated) by the cell. According to the present invention, the step of culturing a cell refers to a step of growing in vitro or in vivo a cell that has been genetically modified to express a recombinant nucleic acid molecule of the present invention.

When the cell is a microorganism or a plant cell to be cultured in vitro, the step of culturing can include culturing the cell in conditions effective to produce the protein. Effective culture conditions include, but are not limited to, effective media, bioreactor, temperature, pH and oxygen conditions that permit protein production. An effective medium refers to any medium in which a cell is cultured to produce an L-galactose dehydrogenase protein of the present invention. Such medium typically comprises an aqueous medium having assimilable carbon, nitrogen and phosphate sources, and appropriate salts, minerals, metals and other nutrients, such as vitamins. Examples of suitable media and culture conditions for both microorganisms and plant cells are discussed in the Examples section. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

When the cell to be cultured is a cell within an organism, such as a higher plant, the step of culturing include conditions effective to maintain the growth of the organism and to allow production of the L-galactose dehydrogenase protein by the cells in the organism. Effective conditions for growing a genetically modified higher plant according to the present invention are also discussed in the Examples section.

The phrase “recovering the protein” refers to collecting the whole fermentation medium containing the protein and or the organism (e.g. a higher plant) containing the protein, and need not imply additional steps of separation or purification. Proteins of the present invention can be purified, if desired, using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. In one embodiment, higher plants which are genetically modified to express or overexpress an L-galactose dehydrogenase protein of the present invention are harvested and can be additionally processed, if desired (i.e., into consumable products).

One embodiment of the present invention relates to an isolated nucleic acid molecule comprising a nucleic acid sequence that is a homologue of a nucleic acid sequence encoding Arabidopsis thaliana L-galactose dehydrogenase. Such a homologue encodes a protein comprising an amino acid sequence that is at least about 40% identical, but is less than 100% identical, to SEQ ID NO:5. Such a homologue encodes a protein having L-galactose dehydrogenase biological activity. In a preferred embodiment, an isolated nucleic acid molecule of the present invention encodes a protein that is at least about 50%, and more preferably, at least about 60%, and more preferably, at least about 70%, and more preferably, at least about 80%, and even more preferably, at least about 90% identical (but less than 100% identical) to SEQ ID NO:5. In one embodiment, an isolated nucleic acid molecule of the present invention comprises a nucleic acid sequence comprising at least about 24 consecutive nucleotides of SEQ ID NO:4, and more preferably, at least about 48 consecutive nucleotides, and more preferably, at least about 96 consecutive nucleotides, and more preferably, at least about 192 consecutive nucleotides, and more preferably, at least about 384 consecutive nucleotides, and even more preferably, 768 consecutive nucleotides of SEQ ID NO:4, but less than 957 consecutive nucleotides of positions 1-957 of SEQ ID NO:4 (i.e., excluding the stop codon at positions 958-960 of SEQ ID NO:4). In another embodiment, an isolated nucleic acid molecule of the present invention comprises a nucleic acid sequence encoding a protein comprising an amino acid sequence which includes at least 8 consecutive amino acids, and more preferably, at least 16 consecutive amino acids, an more preferably, at least 32 consecutive amino acids, and more preferably, at least 64 consecutive amino acids, and even more preferably, at least 128 consecutive amino acids, and even more preferably, at least 256 consecutive amino acids of SEQ ID NO:5, but less than 319 consecutive amino acids of SEQ ID NO:5.

In yet another embodiment, an isolated nucleic acid molecule of the present invention that is less than 100% identical to SEQ ID NO:4 includes a nucleic acid molecule comprising a nucleic acid sequence that hybridizes under low stringency conditions, and more preferably under moderate stringency conditions, and even more preferably under high stringency conditions with a nucleic acid sequence represented by SEQ ID NO:4. Such conditions have been described in detail above. In a preferred embodiment, a recombinant nucleic acid molecule of the present invention includes a nucleic acid molecule comprising a nucleic acid sequence represented by SEQ ID NO:4.

Another embodiment of the present invention relates to plants and other organisms, such as microorganisms, which have been genetically modified to increase the action of L-galactose dehydrogenase. In one embodiment, such a genetic modification increases the biological activity of the L-galactose dehydrogenase. In one embodiment, such an organism, as a result of the increase in L-galactose dehydrogenase action, has increased L-ascorbic acid production, as compared to the organism in the absence of the genetic modification. In another embodiment, the organism, as a result of the increase in L-galactose dehydrogenase action, has increased tolerance to oxidative stress, such as that caused by environmental factors in plants, as compared to the organism in the absence of the genetic modification.

In yet another embodiment, the knowledge of the nucleic acid and amino acid sequence for L-galactose dehydrogenase allows one skilled in the art to produce a plant that has been genetically modified to express a mutated L-galactose dehydrogenase protein (i.e., a homologue of L-galactose dehydrogenase) which is resistant to herbicides that act against the naturally occurring (i.e., endogenous) L-galactose dehydrogenase. In one aspect of this embodiment, the mutated L-galactose dehydrogenase protein has L-galactose dehydrogenase biological activity, in that it is capable of catalyzing the conversion of L-galactose to L-galactono-1,4-lactone. However, in this aspect, the mutant L-galactose dehydrogenase is not targeted by herbicides which act on endogenous L-galactose dehydrogenase. In this manner, genetically engineered plants can survive in the presence of the herbicide, while undesirable plants are damaged or killed. Similarly, using the knowledge of the nucleic acid and amino acid sequence of an L-galactose dehydrogenase as disclosed herein, one of skill in the art is able to identify and/or design compounds that are inhibitors of L-galactose dehydrogenase. Such compounds can be used, for example, in an herbicide which acts on L-galactose dehydrogenase and damages or kills plants that express L-galactose dehydrogenase. The use of herbicides to target L-galactose dehydrogenase and to produce herbicide-resistant plants is discussed in related PCT Publication No. WO 99/33995, supra. The present invention provides the knowledge of the full sequence for L-galactose dehydrogenase, which enables one of skill in the art to produce herbicides and/or herbicide-resistant plants which are sequence-specific.

As used herein, a genetically modified plant (such as a higher plant or microalgae) or microorganism, such as a microalga (Prototizeca, Chlorella), Escherichia coli, or a yeast, is modified (i.e., mutated or changed) within its genome and/or by recombinant technology (i.e., genetic engineering) from its normal (i.e., wild-type or naturally occurring) form. In a preferred embodiment, a genetically modified plant or microorganism according to the present invention has been modified by recombinant technology. Genetic modification of a plant or microorganism can be accomplished using classical strain development and/or molecular genetic techniques, include genetic engineering techniques. Such techniques are generally disclosed herein and are additionally disclosed, for example, in Sambrook et al., (1989), Molecular Cloning: A Laboratory Manual, Cold Spring Harbor Labs Press; Roessler, (1995), Plant Lipid Metabolism, pp. 46-48; Roessler et al., (1994), In: Bioconversion for Fuels, Himmel et al. eds., American Chemical Society, Washington D.C., pp 255-70; and (Horsch, R. B., Fry, J. E., Hoffmann, N. L., Eichholtz, D., Rogers, S. D., Fraley, R. T., (1985), Science 227: 1229-1231). These references are incorporated herein by reference in their entirety.

In some embodiments, a genetically modified plant or microorganism can include a natural genetic variant as well as a plant or microorganism in which nucleic acid molecules have been inserted, deleted or modified, including by mutation of endogenous genes (e.g., by insertion, deletion, substitution, and/or inversion of nucleotides), in such a manner that the modifications provide the desired effect within the plant or microorganism. As discussed above, a genetically modified plant or microorganism includes a plant or microorganism that has been modified using recombinant technology.

As used herein, genetic modifications which result in a decrease in gene expression, an increase in inhibition of gene expression or inhibition of a gene product (i.e., the protein encoded by the gene), a decrease in the function of the gene, or a decrease in the function of the gene product can be referred to as inactivation (complete or partial), deletion, interruption, blockage, down-regulation, or decreased action of a gene. For example, a genetic modification in a gene which results in a decrease in the function of the protein encoded by such gene can be the result of a complete deletion of the gene encoding the protein (i.e., the gene does not exist, and therefore the protein does not exist), an inhibition of the gene transcription such as by using anti-sense technology; a mutation in the gene encoding the protein which results in incomplete or no translation of the protein (e.g., the protein is not expressed), or a mutation in the gene which decreases or abolishes the natural function of the protein (e.g., a protein is expressed which has decreased or no enzymatic activity). An example of inhibition of gene expression using anti-sense is described in Example 3.

Genetic modifications which result in an increase in gene expression or function can be referred to as amplification, overproduction, overexpression, activation, enhancement, addition, up-regulation or increased action of a gene. Additionally, a genetic modification to a gene which modifies the expression, function, or activity of the gene can have an impact on the action of other genes and their expression products within a given metabolic pathway (e.g., by inhibition or competition). In this embodiment, the action (e.g., activity) of a particular gene and/or its product can be affected (i.e., upregulated or downregulated) by a genetic modification to another gene within the same metabolic pathway, or to a gene within a different metabolic pathway which impacts the pathway of interest by competition, inhibition, substrate formation, etc.

In general, a plant or microorganism having a genetic modification that affects L-ascorbic acid production has at least one genetic modification affecting the action of L-galactose dehydrogenase, as discussed above, which results in a change in the biological activity of the gene and its product or in downstream events associated with the action of the gene and its product as compared to a wild-type plant or microorganism grown or cultured under the same conditions. In one embodiment, such a modification changes the ability of the plant or microorganism to produce L-ascorbic acid. According to the present invention, a genetically modified plant or microorganism preferably has an enhanced ability to produce L-ascorbic acid compared to a wild-type plant or microorganism cultured under the same conditions. In another embodiment, the genetically modified plant or microorganism has an enhanced tolerance to oxidative damage compared to a wild-type plant or microorganism grown or cultured under the same conditions.

In a further embodiment, in addition to the modification affecting the action of the L-galactose dehydrogenase, it may be desirable to increase the amount of L-galactose that is available intracellularly in order to increase the output of L-ascorbic acid in the genetically modified plant or microorganism. According to the present invention, the amount of L-galactose that is available intracellularly in a genetically modified plant or microoganism can be increased as compared to wild-type levels, by any suitable method of increasing L-galactose. Such a method can include the delivery of an exogenous supply of L-galactose to the genetically modified plant or microorganism, such as by diffusion, injection, or other method of delivering the L-galactose into the plant or microorganism. As demonstrated in Example 3, the addition of exogenous L-galactose to a genetically modified plant with increased L-galactose dehydrogenase biological activity, increased the production of L-ascorbic acid by the plant Alternatively, L-galactose can be increased in the plant or microorganism by genetically modifying one or more genes involved in the L-ascorbic acid pathway in addition to the L-galactose dehydrogenase, such that the level of L-galactose available prior to the L-galactose dehydrogenase step of the pathway is increased. Genetic modification of additional genes in the L-ascorbic acid pathway can be made using the techniques described herein with regard to L-galactose dehydrogenase.

In one embodiment, genetic modifications are made to an L-ascorbic acid producing organism directly. This allows one to build upon a base of data acquired during prior classical strain improvement efforts, and perhaps more importantly, allows one to take advantage of undefined beneficial mutations that occurred during classical strain improvement. Furthermore, fewer problems are encountered when expressing native, rather than heterologous, genes. In another embodiment of the present invention, discussed in detail below, is to place recombinant nucleic acid molecules encoding a protein having L-galactose dehydrogenase biological activity, or biologically active homologues thereof, which were derived from L-ascorbic acid producing organisms (i.e., higher plants and microalgae) into a plant or microorganism that is more amenable to molecular genetic manipulation, including endogenous L-ascorbic acid producing microorganisms and suitable plants.

It is to be understood that the present invention includes a method comprising the use of a microorganism with an ability to produce commercially useful amounts of L-ascorbic acid in a fermentation process (i.e., preferably an enhanced ability to produce L-ascorbic acid compared to a wild-type microorganism cultured under the same conditions). The present invention also includes a method comprising the use of a genetically modified plant with an ability to produce L-ascorbic acid or esters thereof (i.e., preferably an enhanced ability to produce L-ascorbic acid compared to a wild-type plant cultured or grown under the same conditions). These methods are achieved by the genetic modification of the gene encoding L-galactose dehydrogenase for the production (expression) of a protein having an altered, and preferably, increased, action as compared to the corresponding wild-type protein. Preferably, such genetic modification is achieved by recombinant technology. It will be appreciated by those of skill in the art that production of genetically modified plants or microorganisms having increased L-galactose dehydrogenase biological activity, such as by transformation or transfection of the plant or microorganism with a nucleic acid molecule which encodes a protein having L-galactose dehydrogenase biological activity, can produce many organisms meeting the given functional requirement, albeit by virtue of a variety of different genetic modifications. For example, different random or targeted nucleotide deletions and/or substitutions in a nucleic acid sequence encoding L-galactose dehydrogenase may all give rise to the same phenotypic result (e.g. increased L-galactose dehydrogenase activity). The present invention contemplates any such genetic modification which results in the production of a plant or microorganism having the characteristics set forth herein.

A microorganism to be used in the fermentation method of the present invention is preferably a bacterium, a fungus, or a microalga which has been genetically modified according to the disclosure above. More preferably, a microorganism useful in the present invention is a microalga which is capable of producing L-ascorbic acid, although the present invention includes microorganisms which are genetically engineered to produce L-ascorbic acid using the knowledge of the key components of the pathway and the guidance provided herein. Even more preferably, a microorganism useful in the present invention is an acid-tolerant microorganism, such as microalgae of the genera Prototheca and Chlorella. Acid-tolerant yeast and bacteria are also known in the art. Acid-tolerant microorganisms are discussed in detail below. Particularly preferred microalgae include microalgae of the genera, Prototheca and Chlorella, with Prototheca being most preferred. All known species of Prototheca produce L-ascorbic acid. Production of ascorbic acid by microalgae of the genera Prototheca and Chlorella is described in detail in U.S. Pat. No. 5,792,631, issued Aug. 11, 1998, and in U.S. Pat. No. 5,900,370, issued May 4, 1999, both of which are incorporated herein by reference in their entirety. Preferred bacteria for use in the present invention include, but are not limited to, Azotobacter, Pseudomonas, Agrobacterium and Escherichia, although acid-tolerant bacteria are more preferred. Preferred fingi for use in the present invention include yeast, and more preferably, yeast of the genus, Saccharomyces. A microorganism for use in the fermentation method of the present invention can also be referred to as a production organism. According to the present invention, microalgae can be referred to herein as either microorganisms or as plants.

A preferred plant to genetically modify according to the present invention is preferably a plant suitable for consumption by animals, including humans. More preferably, such a plant is a plant that naturally produces L-ascorbic acid, although other plants can be genetically modified to increase the action of L-galactose dehydrogenase and particularly, to produce L-ascorbic acid, using the guidance provided herein. Particularly preferred higher plants to genetically modify according to the present invention include, but are not limited to plants of the genera Arabidopsis, Pisum, Nicotiana, Solanum, Lactuca, Capsicum, Brassica, Spinacia, Zea, Apium, Daucus, Manihot, banana, Citrus, Pyrus, Malus, Allium, Vicia, Ipomaea, Phaseolus, and Ananas. Preferred microalgae to genetically modify according to the present invention have been described above.

In one embodiment of the present invention, the action of L-galactose dehydrogenase is increased by amplification of the expression (i.e., overexpression) of L-galactose dehydrogenase, including naturally occurring L-galactose dehydrogenase and L-galactose dehydrogenase homologues as discussed previously herein. Overexpression of L-galactose dehydrogenase can be accomplished, for example, by introduction of a recombinant nucleic acid molecule encoding the enzyme. It is preferred that the gene encoding L-galactose dehydrogenase be cloned under control of an artificial promoter. The promoter can be any suitable promoter that will provide a level of enzyme expression required to maintain a sufficient level of L-galactose dehydrogenase and particularly, of L-ascorbic acid production, in the organism. Preferred promoters are constitutive (rather than inducible) promoters, since the need for addition of expensive inducers is therefore obviated. In one embodiment, preferred promoters include tissue-specific promoters (i.e., promoters that drive expression in a specific tissue) for use in higher plants. For example, leaf-specific promoters, tomato fruit-specific promoters and potato tuber-specific promoters are commercially available. The gene dosage (copy number) of a recombinant nucleic acid molecule according to the present invention can be varied according to the requirements for maximum product formation. In one embodiment, the recombinant nucleic acid molecule encoding L-galactose dehydrogenase is integrated into the chromosomes of the microorganism or plant.

Recombinant nucleic acid molecules encoding L-galactose dehydrogenase have been described in detail above, and all previously discussed embodiments of such a recombinant nucleic acid molecule are encompassed for use in a genetically modified organism according to the present invention. Additionally, recombinant nucleic acid molecules encoding L-galactose dehydrogenase can be modified to enhance or reduce the function (i.e., activity) of the L-galactose dehydrogenase protein, as desired to increase L-ascorbic acid production or tolerance to oxidative stress, by any suitable method of genetic modification. For example, a recombinant nucleic acid molecule encoding L-galactose dehydrogenase can be modified by any method for inserting, deleting, and/or substituting nucleotides, such as by error-prone PCR. In this method, the gene is amplified under conditions that lead to a high frequency of misincorporation errors by the DNA polymerase used for the amplification. As a result, a high frequency of mutations are obtained in the PCR products. The resulting gene mutants can then be screened for enhanced substrate affnity and/or enhanced enzymatic activity by testing the mutant genes for the ability to confer increased L-galactose dehydrogenase production and/or increased L-ascorbic acid production or tolerance to oxidative stress onto a test organism, as compared to an organism carrying the non-mutated recombinant nucleic acid molecule.

A nucleic acid molecule can be integrated into the genome of the host cell either by random or targeted integration. Such methods of integration are known in the art. For example, an E. coli strain ATCC 47002 contains mutations that confer upon it an inability to maintain plasmids which contain a ColE1 origin of replication. When such plasmids are transferred to this strain, selection for genetic markers contained on the plasmid results in integration of the plasmid into the chromosome. This strain can be transformed, for example, with plasmids containing the gene of interest and a selectable marker flanked by the 5′- and 3′-termini of the E. coli lacZ gene. The lacZ sequences target the incoming DNA to the lacZ gene contained in the chromosome. Integration at the lacZ locus replaces the intact lacZ gene, which encodes the enzyme β-galactosidase, with a partial lacZ gene interrupted by the gene of interest. Successful integrants can be selected for β-galactosidase negativity.

A genetically modified microorganism can also be produced by introducing nucleic acid molecules into a recipient cell genome by a method such as by using a transducing bacteriophage. The use of recombinant technology and transducing bacteriophage technology to produce several different genetically modified microorganisms of the present invention is known in the art.

Methods for producing a transgenic plant, wherein a recombinant nucleic acid molecule encoding an L-galactose dehydrogenase is incorporated into the genome of the plant, are known in the art. An example of the production of a transgenic plant having L-galactose dehydrogenase is incorporated into its genome is described in the Examples section.

Accordingly, in one embodiment, the present invention includes a method to produce L-ascorbic acid or esters thereof by fermentation of a genetically modified microorganism of the present invention. Such a method includes the step of culturing in a fermentation medium a microorganism having a genetic modification to increase the action of L-galactose dehydrogenase. Preferably, the genetic modification includes transformation or transfection of the microorganism with a recombinant nucleic acid molecule that expresses a protein having L-galactose dehydrogenase biological activity. As discussed in detail above, such a protein can include the overexpression of the endogenous L-galactose dehydrogenase from the L-ascorbic acid pathway of the microorganism, or the recombinant expression of another naturally occurring L-galactose dehydrogenase (e.g., isolated or derived from a different organism), as well as any homologue of a naturally occurring L-galactose dehydrogenase having biological activity. Such a protein is capable of catalyzing the conversion of L-galactose to L-galactono-1,4-lactone.

In the method for production of L-ascorbic acid of the present invention, a microorganism that is genetically modified to increase the action of L-galactose dehydrogenase is cultured in a fermentation medium for production of L-ascorbic acid. An appropriate, or effective, fermentation medium refers to any medium in which a genetically modified microorganism of the present invention, when cultured, is capable of producing L-ascorbic acid. Such a medium is typically an aqueous medium comprising assimilable carbon, nitrogen and phosphate sources. Such a medium can also include appropriate salts, minerals, metals and other nutrients. One advantage of genetically modifying a microorganism as described herein is that although such genetic modifications can significantly alter the production of L-ascorbic acid, they can be designed such that they do not create any nutritional requirements for the production organism. Thus, a minimal-salts medium containing glucose as the sole carbon source can be used as the fermentation medium. The use of a minimal-salts-glucose medium for the L-ascorbic acid fermentation will also facilitate recovery and purification of the L-ascorbic acid product Particularly suitable conditions for culturing a microorganism for the production of L-ascorbic acid are described in PCT Publication No. WO99/64618, published Dec. 16, 1999, incorporated herein by reference in its entirety.

The genetically modified microorganisms of the present invention are engineered to produce significant quantities of extracellular L-ascorbic acid through increased action of L-galactose dehydrogenase and in one embodiment, by additionally providing an increased level of intracellular L-galactose. Extracellular L-ascorbic acid can be recovered from the fermentation medium using conventional separation and purification techniques. For example, the fermentation medium can be filtered or centrifuged to remove microorganisms, cell debris and other particulate matter, and L-ascorbic acid can be recovered from the cell-free supernate by conventional methods, such as, for example, ion exchange, chromatography, extraction, solvent extraction, membrane separation, electrodialysis, reverse osmosis, distillation, chemical derivatization and crystallization. One such example of L-ascorbic acid recovery is provided in U.S. Pat. No. 4,595,659 by Cayle, incorporated herein by reference in its entirety, which discloses the isolation of L-ascorbic acid from an aqueous fermentation medium by ion exchange resin adsorption and elution, which is followed by decoloration, evaporation and crystallition. Further, isolation of the structurally similar isoascorbic acid from fermentation medium by a continuous multi-bed extraction system of anion-exchange resins is described by K. Shimizu, Agr. Biol. Chem. 31:346-353 (1967), which is incorporated herein in its entirety by reference.

Intracellular L-ascorbic acid produced in accordance with the present invention can also be recovered and used in a variety of applications. For example, cells from the microorganisms can be lysed and the ascorbic acid which is released can be recovered by a variety of known techniques. Alternatively, intracellular ascorbic acid can be recovered by washing the cells to extract the ascorbic acid, such as through diafiltration.

Another embodiment of the present invention is a method to produce L-ascorbic acid or esters thereof by growing or culturing a genetically modified plant of the present invention. Such a method includes the step of culturing in a fermentation medium or growing in a suitable environment, such as soil, a plant having a genetic modification to increase the action of L-galactose dehydrogenase. Preferably, the genetic modification includes transformation or transfection of the plant with a recombinant nucleic acid molecule that expresses a protein having L-galactose dehydrogenase biological activity. As discussed in detail above, such a protein can include the overexpression of the endogenous L-galactose dehydrogenase from the L-ascorbic acid pathway of the plant, or the recombinant expression of another naturally occurring L-galactose dehydrogenase (e.g., isolated or derived from a different organism), as well as any homologue of a naturally occurring L-galactose dehydrogenase having biological activity. Such a protein is capable of catalyzing the conversion of L-galactose to L-galactono-1,4-lactone. In one embodiment, the method additionally includes increasing the level of intracellular L-galactose in the genetically modified plant. Methods for increasing L-galactose intracellularly have been described above.

In the method for production of L-ascorbic acid of the present invention, a plant that has a genetic modification to increase the action of L-galactose dehydrogenase is cultured in a fermentation medium or grown in a suitable medium such as soil for production of L-ascorbic acid. An appropriate, or effective, fermentation medium has been discussed in detail above. A suitable growth medium for higher plants includes any growth medium for plants, including, but not limited to, soil, sand, any other particulate media that support root growth (e.g. vermiculite, perlite, etc.) or Hydroponic culture, as well as suitable light, water and nutritional supplements which optimize the growth of the higher plant. The genetically modified plants of the present invention are engineered to produce significant quantities of L-ascorbic acid through increased action of L-galactose dehydrogenase and in one embodiment, additionally through increasing the level of intracellular L-galactose in the plant. The L-ascorbic acid can be recovered through purification processes which extract the L-ascorbic acid from the plant. In a preferred embodiment, the L-ascorbic acid is recovered by harvesting the plant. In this embodiment, the plant can be consumed in its natural state or further processed into consumable products. In one embodiment, the increased L-ascorbic acid production is not intended for use directly as a consumable product, but rather is used to increase the tolerance of plants to oxidative stress which normally damage plants or reduce productivity of harvestable products from the plants. In this embodiment, the increased L-ascorbic acid production by the plant increases the hardiness of the plant so that commercial benefits derived from the plant are also increased.

The invention will be described with reference to the further accompanying drawings FIGS. 1B to 6 in which:

FIG. 1B illustrates the results of SDS PAGE experiments on cell extracts;

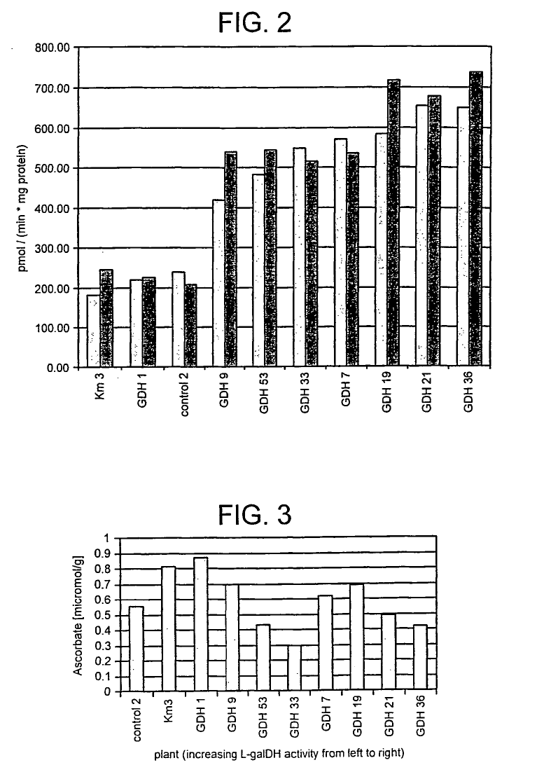

FIG. 2 illustrates L-galactose dehydrogenase activity (pmol min⁻¹ mg protein⁻¹) in the leaves of tobacco (Nicotiana tabacum) plants transformed with the Arabidopsis thaliana L-galDH gene. Each bar represents an individual sample. Results from two samples from 7 independent lines are shown. Control 2=wild type. Km3=plants transformed with vector minus the galDH gene. The galactose dehydrogenase transformed plants are labelled GDH plus the identification number of each line;

FIG. 3 is a graph showing total ascorbate concentration (ascorbate+dehydroascorbate) in the leaves of tobacco (Nicotiana tabacum) plants transformed with the Arabidopsis thaliana. L-galDH gene. Each bar represents the mean value of 3 samples from 7 independent lines. Control 2=wild type. Km3=plants transformed with vector minus the galDH gene. The galactose dehydrogenase transformed plants are labelled GDH plus the identification number of each line. The L-galactose dehydrogenase activities of these lines are shown in FIG. 2;

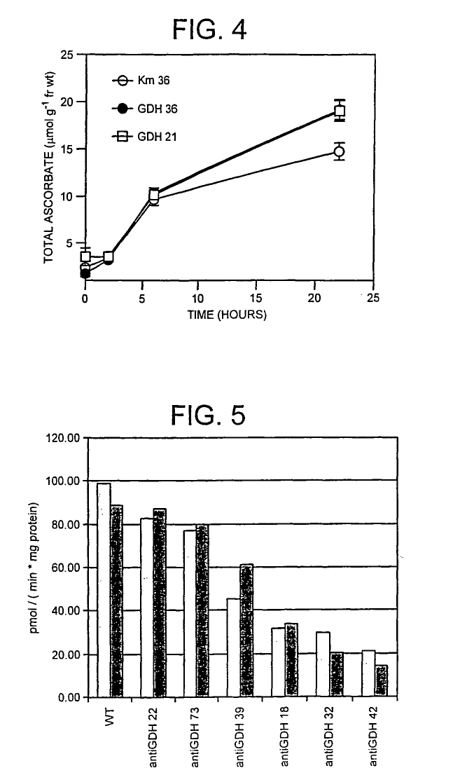

FIG. 4 is a graph illustrating the effect of feeding 10 mM L-galactose to tobacco leaves transformed with L-galactose dehydrogenase (two independent lines: GDH 21 and 36) and plants transformed with a vector minus L-galactose dehydrogenase (Km36) on the total ascorbate (ascorbate+dehydroascorbate) concentration. Leaves were cut into strips 2 mm wide and floated on water containing 10 mM L-galactose for the indicated times. Each point is the mean of 3 replicates plus and minus standard deviation;

FIG. 5 is a graph illustrating L-galactose dehydrogenase activity (pmol min⁻¹ mg protein⁻¹) in the leaves of Arabidopsis thaliana plants transformed with an antisense construct of the L-galDH gene. Each bar represents an individual sample. Results from two samples from 7 independent lines are shown. WT=wild type. The antisense plants are labelled antiGDH plus the identification number of each line; and

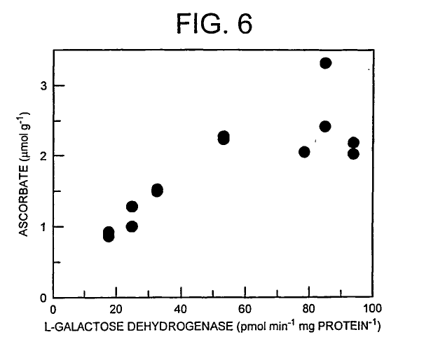

FIG. 6 is a graph illustrating the relationship between the total ascorbate concentration and L-galactose dehydrogenase activity in the leaves of Arabidopsis thaliana plants transformed with an antisense construct of the L-galDH gene. Each data point represents an individual sample. Results from two samples from 7 independent lines are shown.

The following examples are provided for the purposes of illustration and are not intended to limit the scope of the present invention.

### EXAMPLE 1

The following example demonstrates the identification of an Arabidopsis thaliana sequence with homology to the N-terminal amino acid sequence of Pisum sativum L-galactose dehydrogenase.

In order to manipulate ascorbic acid levels within plants and other organisms via the insertion of genetic material it is necessary to identify the nucleotide sequences that encode enzymes involved in ascorbic acid biosynthesis in plants. The following is a description detailing how the present inventors elucidated the nucleotide sequence for L-galactose dehydrogenase.

To determine the nucleotide sequence of the L-galactose dehydrogenase gene it was necessary to utilize the N-terminal amino acid sequence obtained from the protein purified from pea (Pisum sativum) (PCT Publication No. WO 99/33995, published Jul. 8, 1999, incorporated herein by reference in its entirety). It was possible to match this amino acid sequence to other sequences in a number of databases in order to determine the level of homology of any related proteins. A BLAST search was performed by accessing the GenBank database via the National Centre of Biotechnology Information (Briefly, using the amino acid sequence AELRELGRTGLKLGLVGFG (SEQ ID NO:1), which was previously determined from the N-terminus of an isolated Pisum sativum L-galactose dehydrogenase, as described in detail in WO 99/33995, a BLAST 2.0 search was performed using the “blastp” program and the standard default parameters. The results showed that the 19 amino acid N-terminal sequence of P. sativum was 72% identical to a portion of a predicted amino acid sequence from Arabidopis thaliana. This homologous sequence was the N-terminal end of a theoretical protein, identified by the A. thaliana sequencing project, with unknown identity or function (Accession No. CAA20580). The theoretical protein consisted of 319 amino acid residues and therefore would have an estimated molecular weight of approximately 42 kD, which is in accordance with the estimated molecular weight of the individual enzyme sub-units determined from the purified protein from P. sativum (WO 99/33995, ibid.). This theoretical protein is encoded by one of 30 different putative coding regions identified in a 98,124 bp bacterial artificial chromosome (BAC) from Arabidopsis thaliana chromosome 4 (Genbank Accession No. AL031394).

The amino acid sequence obtained from the purified P. sativum protein has therefore enabled the present inventors to identify a gene encoding a homologous putative protein in A. thaliana. Prior to the present invention, there was no knowledge of the identity or function of this putative protein. The following steps detail the cloning strategy employed to show that the gene identified in A. thaliana encodes a protein with L-galactose dehydrogenase activity.

Amplification of the A. thaliana Gene by Reverse Transcriptase-Polymerase Chain Reaction (RT-PCR).

Primer oligonucleotides for RT-PCR with the following sequences were synthesised:

Forward primer: 5′-tca cac atg aeg aaa ata gag ctt cg-3′ (SEQ ID NO:2)

Reverse primer: 5′-ctt ctt tta gtt ctg atg gat tcc act tg-3′ (SEQ ID NO:3)

Total RNA was isolated from young A. thaliana leaves (Sambrook J, Fritsch E F, Maniatis T, 1989: Molecular Cloning: A Laboratory Manual. Cold Spring Harbour, N.Y.). The RNA was used as a template for RT-PCR using the above primers and the following protocol.

The reaction mixture contained:

5 μg of total A. thaliana leaf RNA;

200 U M-MLVReverse Transcriptase (Promega);

30 U RNase inhibitor (Promega);

2 μl 10 mM dNTPs;

1 μl oligo dT(15) primer (0.5 μg/μl);

5 μl 5× reaction buffer (Promega); and,

water to a final volume of 25 μl.

The reaction mixture was exposed to the following temperature regime: 94° C. for 5 min; 30 cycles consisting of a sequence of 58° C. for 1 min, 72° C. for 1.5 min and 94° C. for 1 min; 72° C. for 5 min. After amplification, an aliquot of the reaction mixture was separated by electrophoresis in 1% agarose. The major amplification product, visualized by ethidium bromide staining, had a size of 960 bp.

Cloning of the PCR Product

The 960 bp fragment was excised from the 1% agarose gel and purified with the Qiagen Gel Extraction Kit according to the manufacturer's protocol. The purified DNA was cloned in the pGEM-T cloning vector (Promega) according the protocol of the manufacturer producing the recombinant plasmid pGEM-T-LgalDH which was multiplied in E. coli DH5α (Sambrook J, Fritsch E F, Maniatis T, (1989): Molecular Cloning: A Laboratory Manual. Cold Spring Harbour, N.Y.).

Sequencing of the Cloned cDNA

The recombinant plasmid pGEM-T-LgalDH was purified from E. coli DH5α (Sambrook J, Fritsch E F, Maniatis T, (1989): Molecular Cloning: A Laboratory Manual. Cold Spring Harbour, N.Y.). Sequencing of the insert with universal pUC-M13 sequencing primers was done in both directions by the dideoxy chain termination method (Sanger, F., Nicklen, S and Coulson A R, (1977). Proc. Natl. Acad. Sci USA. 74, 5463-5467) using the Big Dye Sequencing Kit (Perkin Elmer) and an automated DNA sequencer (ABI 377 HT, Perkin Elmer). The sequence of the cloned cDNA is represented herein as SEQ ID NO:4. SEQ ID NO:4 encodes a 319 amino acid sequence represented herein as SEQ ID NO:5.

A BLAST 2.0 search performed by accessing the GenBank database via the National Centre of Biotechnology Information (http://www.ncbi.nlm.min.gov.blast) demonstrated that SEQ ID NO:5 shared 100% identity over all 319 amino acids, as expected, with the putative protein from Arabidopsis thaliana (EMBL Accession No. CAA20580). The next closest homology was a 429 amino acid protein which was 36% identical to SEQ ID NO:5 over 322 amino acids of the 429 amino acid protein, and was identified as Genbank Accession No. AAC43800.1, a protein “similar to phosphotransferase enzyme II and to members of the aldo/keto reductase family” (C. elegans). SEQ ID NO:5 also shared 30% identity over 303 amino acids with a 329 amino acid D-threo-aldose 1-dehydrogenase from Pseudomonas (pir Accession No. JC2405), and 33% identity over 248 amino acids with a 335 amino acid hypothetical protein from Saccharomyces cerevisiae (Accession No. NP_{—}013755.1).

A BLAST 2.0 search also showed that SEQ ID NO:4 shared 100% identity over 303 nucleotides, 100% identity over another 229 nucleotides, 100% identity over another 173 nucleotides, 100% identity over another 136 nucleotides, and 100% identity over another 134 nucleotides, of the BAC clone (EMBL Accession No. AL031394.1) from which the putative protein from Arabidopsis thaliana (EMBL Accession No. CAA20580) was deduced. The next highest score for homology was 96% identity over 27 nucleotides of C. elegans nuclear receptor NHR-3 mRNA (Genbank Accession No. AF083222.1). SEQ ID NO:4 also showed 100% identity over different stretches of 21 nucleotides with Verasper variegatus growth hormone precursor (GH) mRNA (Genbank Accession No. AF086787.1); with a human DNA sequence form clone 798A17 on chromosome 1q24 (emb Accession No. AL031274.1), with Helobdella stagnalis RNA polymerase II second largest subunit (Genbank Accession No. U10337.1); and with D. melanogaster DmTnC 47D mRNA for troponin-C (EMBL Accession No. X76044.1).

### EXAMPLE 2

The following example demonstrates the expression of the A. thaliana cDNA clone in Escherichia coli.

The cDNA cloned from A. thaliana described in Example 1 was subcloned into pBluescript (Stratagene) as follows. pGem-T-LgalDH was digested with ApaI, overhanging ends filled with Klenow enzyme and then digested with PstI. The 960 bp fragment was purified from a 1% agarose gel and ligated into the SmaI/PstI digested pBluescript. E. coli cells were then transformed with this plasmid. Cells harbouring the recombinant plasmid (pBluescript-LgalDH) were isolated and multiplied (Sambrook J, Fritsch E F, Maniatis T, 1989: Molecular Cloning: A Laboratory Manual. Cold Spring Harbour, N.Y.). The plasmid was then isolated and the 960 bp cDNA excised by digestion with BamHI and EcoRI restriction enzymes. The resulting fragment was purified from a 1% agarose gel and cloned into the BamHI-EcoRI site of the pRSETB vector to produce pRSETB-LgalDH. E. coli cells (strain BL21(DE3)lysS) were then transformed with the pRSETB-LgalDH plasmid. Transformed cells were exposed to 1 mM isopropyl b-D-thiogalactopyranoside (IPTG) and at intervals, the cells were harvested by centrifugation. Harvested cells were suspended in 50 mM tris-HCl buffer at pH 7.5 containing 20% (v/v) glycerol, 2 mM dithiothreitol, 0.5% (v/v) Triton X-100 and 1 mM ethylenediaminetetraacetic acid, and subjected to a freeze-thaw cycle in liquid nitrogen followed by sonication. The cell debris was removed by centrifugation at 1300 rpm for 10 minutes and the supernatant assayed for L-galactose dehydrogenase activity as follows.

The reaction mixture contained (in 1 ml) 50 mM tris-HCl buffer, pH 7.5, 0.1 mM nicotinamide adenine dinucleotide (NAD), 2 mM L-galactose and 20 μl E. coli supernatant The rate of L-galactose-dependent NAD reduction was measured by monitoring the increase in absorbance of the reaction mixture at 340 nm over a period of 200 seconds. Untransformed E. coli cells and those transformed with pRSETB had no detectable L-galactose dehydrogenase activity. Cells transformed with pRSETB-LgalDH had a low level of activity, which was rapidly increased after inducing expression of the cloned cDNA by adding 1 mM IPTG (FIG. 1). Cell extracts with L-galactose dehydroganase activity had a novel polypeptide of 40 kD which could be visualized by staining with coomassie blue after separation by sodium dodecyl sulphate-polyacrylamide gel electrophoresis (SDS-PAGE) (FIG. 1B). This polypeptide was not present in untransformed cells.

These results show that the 960 bp cDNA isolated from A. thaliana encodes a protein with L-galactose dehydrogenase activity which is of similar size to the L-galactose dehydrogenase enzyme originally purified from P. sativum (WO 99/33995, ibid.). Further purification of the enzyme activity was carried out by Ni2+ affinity chromatography (which binds the poly-histidine tag of the recombinant protein) and size exclusion chromatography. Nickel affinity chromatography was carried out by passing an E. coli extract, prepared as described above, through a column containing NTA-agarose (Novagen “His-Bind Resin”). 5 ml of the resin were washed with 30 ml H₂O, charged with 30 ml NiSO₄ washed with 30 ml H₂O and equilibrated with 30 ml 50 mM Na-phosphate pH 8, 300 mM NaCl. The E. coli pellet was suspended in 50 mM Na-phosphate pH 8, 300 mM NaCl and sonicated. The suspension was centrifuged again (9000 g, 20 min). The supernatant was stirred with the Ni-resin on ice for 1 hour. The suspension was transferred into a column. After settling down the column was washed with 250 ml 50 mM Na-phosphate pH 6 containing 300 mM NaCl an d 10% glycerol. Ni-bound protein was eluted with 500 mM imidazole/50 mM Na-phosphate at pH 6 containing 300 mM NaCl. Fractions were collected and assayed for L-galactose dehydrogenase activity. The active fractions were pooled and proteins precipitated by addition of ammonium sulphate to 85% saturation. After centrifugation at 9000 g for 15 minutes the pellet was suspended in 2 ml 25 mM tris-HCl buffer pH 7 containing 150 mM NaCl. This sample was then subjected to size exclusion chromatography on a Superdex 200 column (Amersham-Pharmacia Biotech) using 25 mM tris-HCl buffer pH 7 containing 150 mM NaCl as eluent Fractions (1 ml) were collected and two peaks of activity corresponding to molecular weights of 42.4 and 87.5 kD were detected. SDS-PAGE of each of these peaks of L-galactose dehydrogenase activity showed the presence of a single polypeptide with an estimated molecular mass of 42.2 kD (data not shown). The results show that the recombinant gene encodes a polypeptide of 42 kD with L-galactose dehydrogenase activity. Under the chromatography conditions employed this protein exists as a mixture of monomers and dimers.

### EXAMPLE 3

The following example describes the production of transgenic plants that over-express L-galactose dehydrogenase.

L-Galactose dehydrogenase has been identified as an enzyme involved in biosynthesis of L-ascorbic acid in plants (Wheeler et al., 1998). The A. thaliana cDNA encoding L-galactose dehydrogenase can therefore be overexpressed in transgenic plants. In addition, the nucleic acid molecule encoding L-galactose dehydrogenase could be overexpressed in any other transgenic organism to increase ascorbic acid concentration or to introduce the capacity for ascorbic acid production into that organism. As an example, Arabidopsis L-galactose dehydrogenase was expressed in tobacco plants using the following procedure.

Cloning of the gene in the binary vector pGPTV-KAN (Becker D, Kemper E, Schell J and Masterson R, 1992. Plant Mol. Biol. 20: 1195-1197)

The reporter gene uid A of the vector pGPTV-KAN was replaced by the CaMV 35S promoter. The cDNA encoding the Arabidopsis thaliana L-galactose dehydrogenase was inserted in the SmaI site downstream of the CaMV 35S promoter. The vector pGPTV-KAN-P35S was digested with SmaI and dephosphorylated with calf intestinal alkaline phosphatase. The linearized vector was purified from a preparative gel of 1% low melting point agarose by extraction with phenol/chloroform and precipitation with Na-acetate/ethanol. The insert was prepared by digestion of the plasmid pGem-T-LgalDH with SacI and PstI. Overhanging ends were filled with Klenow enzyme and the LgalDH fragment purified from a 1% agarose gel. Vector and insert were ligated with T4 DNA ligase according the instructions of the manufacturer. After transformation of E. coli DH5a the sense and antisense clones were identified by restriction analysis and sequencing.

Transformation of Escherichia coli and Agrobacterium tumefaciens.

E. coli DH5a was transformed with the ligated DNA by electroporation (Dower, W. J., Miller, J. F., Ragsdale, C. W., 1983, Nucl. Acids Res. 16: 6127-6145). A. tumefaciens LBA4404 was transformed with purified plasmid DNA as described by Holsters et al. (Holsters, M., de Waele, D., Depicker, A., Messens, E., van Montagu, M., Schell, J., (1978), Mol. Gen. Genet. 163: 181-187, incorporated herein by reference in its entirety). Briefly, a pre-culture of the recipient bacteria (A. tumefaciens LBA4404) was grown overnight at 28° C. in YEP medium (An G, Ebert P R, Mitra A and Hearst J E (1988) Plant Mol. Biol. Manual (Gelvin S B and Schilperoort R A, ed.). Kluwer Acad. Publ., A3: 1-19). The culture was washed and concentrated to approximately 10¹⁰-5×10¹⁰ cells/ml, mixed with the pGPTV-LgalDH plasmid, and frozen in liquid nitrogen. The cultures were then thawed for 25 min. at 37° C., diluted 5-fold in YEB medium, and incubated at 28° C. to allow phenotypic expression and plated on YEB plates for selection of positive transformants.

Transformation of Tobacco Plants.

Nicotiana tabacum SRI was transformed by the leaf disc method (Horsch, R. B., Fry, J. E., Hoffmann, N. L., Eichholtz, D., Rogers, S.D., Fraley, R. T., 1985, Science 227: 1229-1231, incorporated herein by reference in its entirety). Briefly, discs (6 mm diameter) were punched from surface sterilized leaves of Nicotiana tabacum SRI and were submerged in a culture of A. tumefaciens grown overnight in luria broth at 28° C. After gentle shaking to ensure that all of the edges were infected, the discs were blotted dry and incubated upside down on plates with MS medium (Murashige T. and Skoog F. 1962. Physiol. Plant. 15: 473-479) containing 3% sucrose, 2 mg/l benzylaminopurine and 0.05 mg/l naphtaleneacetic acid. After 2 to 3 days, the discs were transferred to petri plates containing the same medium but without feed cells or filter papers and containing carbenicillin (500 μg/ml) and kanamycin (100 μg/ml). After 24 weeks, shoots that developed were excised from calli and transplanted to appropriate root-inducing medium containing carbenicillin (500 μg/ml) and kanamycin (100 μg/ml). Rooted plantlets were transplanted to soil as soon as possible after roots appeared. Plants that expressed the Arabidopsis thaliana L-galactose dehydrogenase transgene were identified by Northern blots (Sambrook J, Fritsch E F, Maniatis T, 1989: Molecular Cloning: A Laboratory Manual. Cold Spring Harbour, N.Y.) using a non-radioactive detection system (The DIG System User's Guide for Filter Hybridization, Boehringer Mannheim 1995). 8 of these lines were chosen, allowed to self-pollinate and seed was collected. The seed was then sown on kanamycin selection medium as above. 15 kanamycin resistant seedlings of each of 8 independent lines were then grown in a glasshouse, along with controls consisting of non-transformed plants and a line transformed with vector lacking the L-galDH transgene. At the 5 leaf stage, plants were assayed for transgene expression by Northern blots, by immunoblots (Western blots) with L-galactose dehydrogenase antibody raised against recombinant Arabidopsis L-galactose dehydrogenase) and by activity of L-galactose dehydrogenase activity in leaf extracts. For immunodetection of L-galactose dehydrogenase protein by Western blotting, proteins extracted from tobacco leaves with 50 mM tris-HCl buffer at pH 7.5 containing 20% (v/v) glycerol, 2 mM dithiothreitol, 1 mM ethylenediaminetetraacetic acid, 1 mM aminocaproic acid, 1 mM benzamidine, 1 mM phenylmethylsulfonyl fluoride and 1% (w/v) polyvinylpolypyrrolidone were separated by SDS-PAGE electrophoresis on a 10% polyacrylamide gel. They were then transferred to a PVDF membrane in a semidry blotting apparatus using 0.1 M Tris, 0.192 M glycine, 5% methanol as transfer buffer. The immunodetection of L-galDH was performed with an antibody specific against recombinant L-galDH (expressed with pRSETB-L-galDH) and the ECL Western Blotting System (Amersham Pharmacia Biotech).

FIG. 2 shows the L-galactose dehydrogenase activity extracted from leaves of 8 lines transformed tobacco plants (GDH1, 7, 9, 19, 21, 33, 36, and 53) compared with the untransformed control (Control 2) and the control line transformed with a vector lacking the L-galDH transgene (Km3). To measure L-galDH activity leaves (0.1 g fresh weight) were homogenised in 0.5 ml 50 mM tris-HCl buffer at pH 7.5 containing 20% (v/v) glycerol, 2 mM dithiothreitol, 1 mM ethylenediaminetetraacetic acid, 1 mM aminocaproic acid, 1 mM benzamidine, 1 mM Phenylmethylsulfonyl fluoride and 1% (w/v) polyvinylpolypyrrolidone. The homogenate was centrifuged at 12,000 g for 2 minutes and the supernatant used for enzyme assay. L-GalDH was measured by determining the ability of leaf extracts to convert L-[¹⁴C]galactose to L-[¹⁴C]galactono-1,4-lactone in the presence of nicotinamide adenine dinucleotide (NAD). The reaction mixture contained 50 mM Tris pH 7.5 (10 ml), 4 mM NAD (2 ml), 0.02 mCi ¹⁴C-L-galactose (specific activity 55 mCi/mmol) (2 ml) and plant extract (6 ml). It was incubated at room temperature and stopped after 20 min. by adding of 20 ml ethanol. L-[¹⁴C]galactose to L-[¹⁴C]galactono-1,4-lactone in the reaction mixture were then separated by thin layer chromatography (TLC) on silica plates soaked for 20 min. in 0.3 M NaH₂PO₄ and dried. The mobile phase was acetone/butanol/water (8/1/1 by volume). After drying the amount of ¹⁴C in L-galactose to L-galactono-1,4-lactone plates was determined by scanning the plates with a radioactivity detector (Berthold Analytical, Gaithersburg, Md., USA). L-galactose and L-galactono-1,4-lactone identified by reference to standards. The transformed plants contained a range of L-galDH activities from similar to untransformed controls (GDH1) to 3.5 times higher (GDH19, 21 and 36). Measurements of the amount of L-galDH protein by Western blots and of L-galDH mRNA by Northern blots as described in the previous paragraph showed that those lines with low L-galDH activity had mRNA and L-galDH protein levels similar to unstransformed plants, while the plants with high L-galDH activity had correspondingly higher L-galDH mRNA and L-galDH protein levels (data not shown). The results show that transformation of tobacco plants with the A. thaliana L-galDH gene causes an increase in L-galDH activity. The same plants were then analysed for ascorbate plus dehydroascorbate content after homogenising the leaves in 6% (w/v) trichloroacetic acid (0.2 g fresh weight leaf in 0.5 ml). The extracts were centrifuged at 12,000 g for 2 minutes and assayed for total ascorbate (ascorbate plus dehydroascorbate, Kampfenkel, K., Van Montagu, M., Inze, D. (1995) Analytical Biochemistry 225: 165-167). The results show that the ascorbate content was unchanged by over-expression of L-galDH in tobacco (FIG. 3). However, it was demonstrated that the introduced enzyme is potentially active in vivo by supplying tobacco leaves with L-galactose. These transformed L-galactose to ascorbate faster than vector-only transformants (FIG. 4).

### EXAMPLE 4

Arabidopsis thaliana plants were transformed with the same pGPTV-LgalDH vector as described above, except that the L-galDH was in antisense orientation, via Agrobacterium-mediated transformation of flower buds (S. J. Clough and A. F. Bent (1998) The Plant Journal 16, 735-743). Kanamycin-resistant seedlings were selected by growth on agar containing 0.5×MS medium (Murashige T and Skoog F (1962) Physiol. Plant. 15: 473-479), 1% sucrose and kanamycin (100 μg ml⁻¹). About 20 T1 kanamycin-resistant transformants were produced. These were allowed to self-pollinate and seed was collected. The seeds were then germinated on the kanamycin selection medium and resistant plants from 6 independent lines were transferred to a glasshouse. Leaves were collected from the plants just prior to flowering and they were analysed for expression of L-galDH mRNA by northern blotting, L-galDH protein by immunoblotting, L-galDH enzyme activity and ascorbate content by the same methods as described in Example 3. L-GalDH activity varied from close to wild type (untransformed plants) to 22% of the wild type in lines antiGDH 32 and antiGDH 42 (FIG. 5). The levels of galDH mRNA and protein in these two lines were also reduced by antisense expression of L-galDH (data not shown). Ascorbate concentration in the leaves of these same lines was reduced relative to the control and comparison of all the lines showed a progressive decrease in ascorbate content as L-galDH activity drops below 50% of wild type (FIG. 6). These results provide unequivocal evidence that L-galDH is involved in ascorbate biosynthesis in plants.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims:

1. An isolated protein having L-galactose dehydrogenase biological activity, wherein the protein comprises an amino acid sequence selected from the group consisting of:
a) an amino acid sequence selected from the group consisting of SEQ ID NO:1 and SEQ ID NO:5; and
b) a homologue of the amino acid sequence of (a), wherein the homologue is at least 40% identical to SEQ ID NO:5.

2. An isolated protein according to claim 1, wherein the protein comprises an amino acid sequence that is at least 60% identical to SEQ ID NO:5.

3. An isolated protein according to claim 2, wherein the protein comprises an amino acid sequence that is at least 70% identical to SEQ ID NO:5.

4. An isolated protein according to claim 3, wherein the protein comprises an amino acid sequence that is at least 80% identical to SEQ ID NO:5.

5. An isolated protein according to any preceding claim, wherein the protein comprises an amino acid sequence comprising at least 8 consecutive amino acids of SEQ ID NO:5.

6. An isolated protein according to any one of claims 1 to 5, wherein the protein is encoded by a nucleic acid molecule comprising a nucleic acid sequence that hybridizes under moderate stringency conditions to SEQ ID NO:4.

7. An isolated protein according to claim 6, wherein the protein is encoded by a nucleic acid molecule comprising a nucleic acid sequence that hybridizes under high stringency conditions to SEQ ID NO:4.

8. An isolated protein according to claim 1, wherein the protein is encoded by a nucleic acid molecule comprising SEQ ID NO:4.

9. An isolated protein according to any preceding claim, wherein the protein is NAD or NADP-dependent.

10. An isolated protein according to any preceding claim, wherein the protein oxidises carbon atom 1 of L-galactose.

11. A recombinant nucleic acid molecule comprising an expression vector operatively linked to a nucleic acid molecule comprising a nucleic acid sequence encoding a protein according to any one of claims 1 to 10.

12. A recombinant nucleic acid molecule according to claim 11, wherein the nucleic acid molecule comprises a nucleic acid sequence that is less than 100% identical to SEQ ID NO:4.

13. A recombinant nucleic acid molecule according to claim 11 or 12, wherein the nucleic acid molecule comprises a nucleic acid sequence comprising at least 24 consecutive nucleotides of SEQ ID NO:4.

14. A recombinant nucleic acid molecule according to any one of claims 11 to 13, wherein said nucleic acid molecule comprises a nucleic acid sequence that is at least 97% identical to SEQ ID NO:4 over at least 27 consecutive nucleotides of SEQ ID NO:4.

15. A recombinant nucleic acid molecule according to any one of claims 11 to 14, wherein the expression vector is a plant expression vector.

16. A recombinant nucleic acid molecule according to any one of claims 11 to 14, wherein the expression vector is suitable for use in a microorganism.

17. A recombinant nucleic acid molecule according to claim 16, wherein the microorganism is a microalga.

18. A recombinant nucleic acid molecule according to claim 16, wherein the microorganism is a bacterium.

19. A recombinant nucleic acid molecule according to claim 16, wherein the microorganism is a fungus.

20. A recombinant nucleic acid molecule according to claim 19, wherein the fungus is a yeast.

21. A method for producing a protein having L-galactose dehydrogenase biological activity, comprising culturing an isolated cell that has been genetically modified to express a recombinant nucleic acid molecule according to any one of claims 11 to 20 under conditions whereby the protein encoded by the recombinant nucleic acid molecule is expressed by the cell.

22. An isolated nucleic acid molecule comprising a nucleic acid sequence encoding an isolated protein according to any one of claims 1 to 10.

23. An isolated nucleic acid molecule according to claim 22, wherein the nucleic acid molecule encodes a protein comprising at least 32 consecutive amino acids of an amino acid sequence represented by SEQ ID NO:5.

24. An isolated nucleic acid molecule according to claim 23, wherein the nucleic acid molecule encodes a protein comprising at least 64 consecutive amino acids of an amino acid sequence represented by SEQ ID NO:5.

25. A plant which has a genetic modification to increase the action of L-galactose dehydrogenase.

26. A plant according to claim 25, wherein the genetic modification increases the biological activity of L-galactose dehydrogenase.

27. A plant according to claim 25 or 26, in which L-galactose is increased intracellularly in the plant.

28. A plant according to claim 25, 26 or 27 wherein the plant has increased L-ascorbic acid production as compared to the plant in the absence of the genetic modification.

29. A plant according to any one of claims 25 to 28 wherein the plant has increased tolerance to oxidative stress as compared to the plant in the absence of the genetic modification.

30. A plant according to any one of claims 25 to 29, wherein the plant is genetically modified to express a recombinant nucleic acid molecule that encodes a protein having L-galactose dehydrogenase biological activity.

31. A plant according to claim 30, wherein the recombinant nucleic acid molecule comprises a nucleic acid molecule according to any one of claims 11 to 20.

32. A plant according to any one of claims 25 to 31, wherein the plant is a microalga.

33. A plant according to claim 32, wherein the plant is selected from the group consisting of microalgae of the genera Prototheca and Chlorella.

34. A plant according to any one of claims 25 to 31, wherein the plant is a higher plant.

35. A plant according to any one of claims 25 to 34, wherein the plant or a portion thereof is consumable.

36. A microorganism for producing ascorbic acid or esters thereof, wherein the microorganism has a genetic modification to increase the action of L-galactose dehydrogenase.

37. A microorganism according to claim 36, wherein the microorganism is genetically modified to express a recombinant nucleic acid molecule that encodes a protein having L-galactose dehydrogenase biological activity.

38. A microorganism according to claim 36 or 37, wherein the recombinant nucleic acid molecule comprises a nucleic acid molecule according to any one of claims 12 to 21.

39. A microorganism according to any one of claims 36 to 38, wherein the microorganism is selected from the group consisting of bacteria, fungi and microalgae.

40. A method for producing ascorbic acid or esters thereof in a plant, comprising growing a plant according to any one of claims 25 to 35.

41. A method for producing ascorbic acid or esters thereof in a microorganism, comprising culturing a microorganism according to any one of claims 35 to 39.

42. A method according to claim 40 or claim 41, wherein said genetic modification increases the biological activity of L-galactose dehydrogenase.


SEQUENCE LISTING (7 sequences)

SEQ ID NO: 1 - protein, 19 aa, Pisum sativum
AELRELGRTGLKLGLVGFG

SEQ ID NO: 2 - DNA, 26 bp, Artificial sequence
  primer
tcacacatgacgaaaatagagcttcg

SEQ ID NO: 3 - DNA, 29 bp, Artificial sequence
  primer
cttcttttagttctgatggattccacttg

SEQ ID NO: 4 - DNA, 960 bp, Arabidopsis thaliana
  CDS (1)..(960)
atgacgaaaatagagcttcgagctttggggaacacagggcttaaggttagcgccgttggttttggtgcctctccgctcggaagtgtcttcggtccagtcgccgaagatgatgccgtcgccaccgtgcgcgaggctttccgtctcggtatcaacttcttcgacacctccccgtattatggaggaacactgtctgagaaaatgcttggtaagggactaaaggctttgcaagtccctagaagtgactacattgtggctactaagtgtggtagatataaagaaggttttgatttcagtgctgagagagtaagaaagagtattgacgagagcttggagaggcttcagcttgattatgttgacatacttcattgccatgacattgagttcgggtctcttgatcagattgtgagtgaaacaattcctgctcttcagaaactgaaacaagaggggaagacccggttcattggtatcactggtcttccgttagatattttcacttatgttcttgatcgagtgcctccagggactgtcgatgtgatattgtcatactgtcattacggcgttaatgattcgacgttgctggatttactaccttacttgaagagcaaaggtgtgggtgtgataagtgcttctccattagcaatgggcctccttacagaacaaggtcctcctgaatggcaccctgcttcccctgagctcaagtctgcaagcaaagccgcagttgctcactgcaaatcaaagggcaagaagatcacaaagttagctctgcaatacagtttagcaaacaaggagatttcgtcggtgttggttgggatgagctctgtctcacaggtagaagaaaatgttgcagcagttacagagcttgaaagtctggggatggatcaagaaactctgtctgaggttgaagctattctcgagcctgtaaagaatctgacatggccaagtggaatccatcagaactaa

SEQ ID NO: 5 - protein, 319 aa, Arabidopsis thaliana
MTKIELRALGNTGLKVSAVGFGASPLGSVFGPVAEDDAVATVREAFRLGINFFDTSPYYGGTLSEKMLGKGLKALQVPRSDYIVATKCGRYKEGFDFSAERVRKSIDESLERLQLDYVDILHCHDIEFGSLDQIVSETIPALQKLKQEGKTRFIGITGLPLDIFTYVLDRVPPGTVDVILSYCHYGVNDSTLLDLLPYLKSKGVGVISASPLAMGLLTEQGPPEWHPASPELKSASKAAVAHCKSKGKKITKLALQYSLANKEISSVLVGMSSVSQVEENVAAVTELESLGMDQETLSEVEAILEPVKNLTWPSGIHQN

SEQ ID NO: 6 - DNA, 29 bp, Artificial sequence
  primer
gattcacccatgacgaaaatagagcttcg

SEQ ID NO: 7 - DNA, 29 bp, Artificial sequence
  primer
cttcttttagttctgatggattccacttg